(19) United States
(12) Patent Application Publication
(10) Pub. No.: US 2017/0167998 A1
(43) Pub. Date: Jun. 15, 2017

(54) SENSOR FIBERS AND METHODS OF MANUFACTURING THE SAME

(71) Applicant: Electronics and Telecommunications Research Institute, Daejeon (KR)

(72) Inventor: Hyung-Kun LEE, Daejeon (KR)

(21) Appl. No.: 15/372,665

(22) Filed: Dec. 8, 2016

(30) Foreign Application Priority Data
Dec. 9, 2015 (KR) 10-2015-0175265

(51) Int. Cl. G01N 27/12; G01N 33/00; B05D 1/18

(52) U.S. Cl. CPC G01N 27/125; B05D 1/18; G01N 33/0027

(57) ABSTRACT

Disclosed are a sensor fiber and a method of manufacturing the same. The method of manufacturing a sensor fiber comprises providing a mixture solution including a detection member, submerging a core fiber in the mixture solution, and coating the detection member on a surface of the core fiber by stirring the mixture solution in which the core fiber is submerged. The detection member comprises a transition metal chalcogenide.

### CROSS-REFERENCE TO RELATED APPLICATIONS

This U.S. nonprovisional patent application claims priority under 35 U.S.C §119 of Korean Patent Application 10-2015-0175265 filed on Dec. 9, 2015 entire contents of which are hereby incorporated by reference.

### BACKGROUND

The present inventive concepts relate to a sensor fiber and a method of manufacturing the same and, more particularly, to a sensor fiber and a method of manufacturing the same including transition metal chalcogenide.

In addition to the case where gas is used as traditional energy source, gas sensors are widely employed along with the increase of gas applications. Conventional gas sensors are mainly used to detect toxic or explosive gas, but extensive technologies are recently developed to detect and utilize gases in many fields such as health care, environmental pollution monitoring, industry safety, home appliances and smart home, food and agriculture, national defense and terror, and so on. In order to exactly and sensitively detect samples including therein target materials in many fields, detection techniques have been variously developed. The current commercialized gas sensors are of an optical type, an electrochemical type, a semiconductor type, a catalytic combustion type, a surface acoustic wave type, and so on. The gas sensors mentioned above are mostly implemented on inflexibly tough substrates. However, it has recently been required to develop flexible gas sensors, in particular fabric shaped gas sensors, applicable to wearable devices or flexible electronic devices.

### SUMMARY

Example embodiments of the present inventive concepts provide a sensor fiber that detects gas and a method of fabricating the same.

An object of the present inventive concept is not limited to the above-mentioned one, other objects which have not been mentioned above will be clearly understood to those skilled in the art from the following description.

According to exemplary embodiments of the present inventive concepts, a method of manufacturing a sensor fiber may comprise: providing a mixture solution including a detection member; submerging a core fiber in the mixture solution; and coating the detection member on a surface of the core fiber by stirring the mixture solution in which the core fiber is submerged.

In some exemplary embodiments, the detection member may comprise a transition metal chalcogenide.

In some exemplary embodiments, the transition metal chalcogenide may comprise molybdenum sulfide (MoS₂) or tungsten sulfide (WS₂).

In some exemplary embodiments, the core fiber may comprise at least one of a polymer fiber, a natural fiber, a metal fiber, an inorganic fiber, or a composite fiber thereof.

In some exemplary embodiments, the core fiber may be provided in plural. The plurality of core fibers may be interwoven into a woven fabric or may be braided into a braided fabric.

In some exemplary embodiments, the detection member may further comprise at least one of a metal nanoparticle, a graphene, a graphene derivate, a metal oxide, or a conductive polymer.

In some exemplary embodiments, the method may further comprise, before submerging the core fiber in the mixture solution, coating an adhesive member on the surface of the core fiber. The detection member may be coated on a surface of the adhesive member when the mixture solution is stirred.

In some exemplary embodiments, the step of coating the adhesive member on the surface of the core fiber may comprise: providing an adhesive solution that includes the adhesive member; submerging the core fiber in the adhesive solution; and coating the adhesive member on the surface of the core fiber by stirring the adhesive solution in which the core fiber is submerged.

In some exemplary embodiments, the adhesive member may comprise polydopamine, BSA (Bovine Serum Albumin), beta-amyloid, polylysine, chitosan, or any other polymer adhesives.

According to exemplary embodiments of the present inventive concepts, a sensor fiber may comprise: a core fiber; an adhesive member surrounding a surface of the core fiber; and a detection member surrounding a surface of the adhesive member.

In some exemplary embodiments, the detection member may comprise a transition metal chalcogenide.

In some exemplary embodiments, the transition metal chalcogenide may comprise molybdenum sulfide (MoS₂) or tungsten sulfide (WS₂).

In some exemplary embodiments, the detection member may further include at least one of a metal nanoparticle, a graphene, a graphene derivate, a metal oxide, or a conductive polymer.

In some exemplary embodiments, wherein the adhesive member may comprise polydopamine, BSA (Bovine Serum Albumin), beta-amyloid, polylysine, chitosan, or any other polymer adhesives.

In some exemplary embodiments, the core fiber may comprise at least one of a polymer fiber, a natural fiber, a metal fiber, an inorganic fiber, or a composite fiber thereof.

### BRIEF DESCRIPTION OF THE DRAWINGS

FIG. 1 is a partial perspective view for explaining an example of a sensor fiber according to exemplary embodiments of the present inventive concepts.

FIG. 2 is a cross-sectional view for explaining the sensor fiber of FIG. 1.

FIG. 3 is a cross-sectional view for explaining other example of a sensor fiber according to exemplary embodiments of the present inventive concepts.

FIG. 4 is a schematic diagram for explaining a fabric-based gas sensor system using a sensor fiber according to exemplary embodiments of the present inventive concepts.

FIG. 5 is a flow chart for explaining a method of manufacturing a sensor fiber according to exemplary embodiments of the present inventive concepts.

FIG. 6 is a SEM photograph of a sensor fiber manufactured by an experimental example.

FIG. 7 is a graph showing NO₂ detection result of a sensor fiber manufactured by an experimental example.

### DETAILED DESCRIPTION OF EMBODIMENTS

In order to sufficiently understand the configuration and effect of the present invention, embodiments of the present invention will be described with reference to the accompanying drawings. It should be noted, however, that the present invention is not limited to the following exemplary embodiments, and may be implemented in various forms. Rather, the exemplary embodiments are provided only to disclose the present invention and let those skilled in the art fully know the scope of the present invention. One of ordinary skill in the art will understand that the present inventive concept may be carried out in any suitable environment. Like reference numerals refer to like elements throughout the specification.

The terminology used herein is for the purpose of describing particular embodiments only and is not intended to limit the present invention. As used herein, the singular forms are intended to include the plural forms as well. It will be understood that the terms “comprises”, and/or “comprising” specify the presence of stated elements, steps, operations, and/or devices, but do not preclude the presence or addition of one or more other elements, steps, operations, and/or devices.

It will be understood that when a surface (or layer) is referred to as being disposed on a substrate, it can be directly disposed on the substrate or other surface (or layer), or intervening elements may be present.

It will be understood that, although the terms “first”, “second”, “third”, etc. may be used herein to describe various regions and sections (or layers), these regions and sections should not be limited by these terms. These terms are only used to distinguish one region or section (or layer) from another region or section (or layer). Accordingly, a first section referred in some embodiments may also be referred to as a second section in other embodiments. The exemplary embodiments explained and illustrated herein include complementary embodiments thereof. Like reference numerals refer to like elements throughout the specification.

In addition, the embodiments in the detailed description will be discussed with reference to sectional and/or plan views as ideal exemplary views of the present invention. In the drawings, thicknesses of layers and regions are exaggerated for effectively explaining the technical contents. Accordingly, variations from the shapes of the illustrations as a result of manufacturing techniques and/or tolerances are to be expected. Thus, exemplary embodiments should not be construed as limited to the shapes of regions illustrated herein but are to include deviations in shapes that result from manufacturing process. For example, an etching region illustrated as rectangle will typically have rounded or curved features. Accordingly, regions exemplarily illustrated in the drawings are schematic in nature, and their shapes are intended to exemplarily disclose actual shapes of a region of a device and are not intended to limit the scope of the scope of the present invention.

Unless otherwise defined, all terms used herein have the same meaning as commonly understood by one of ordinary skill in the art to which exemplary embodiments of the present invention belong.

It will be hereinafter described about exemplary embodiments of the present inventive concept with reference to the accompanying drawings.

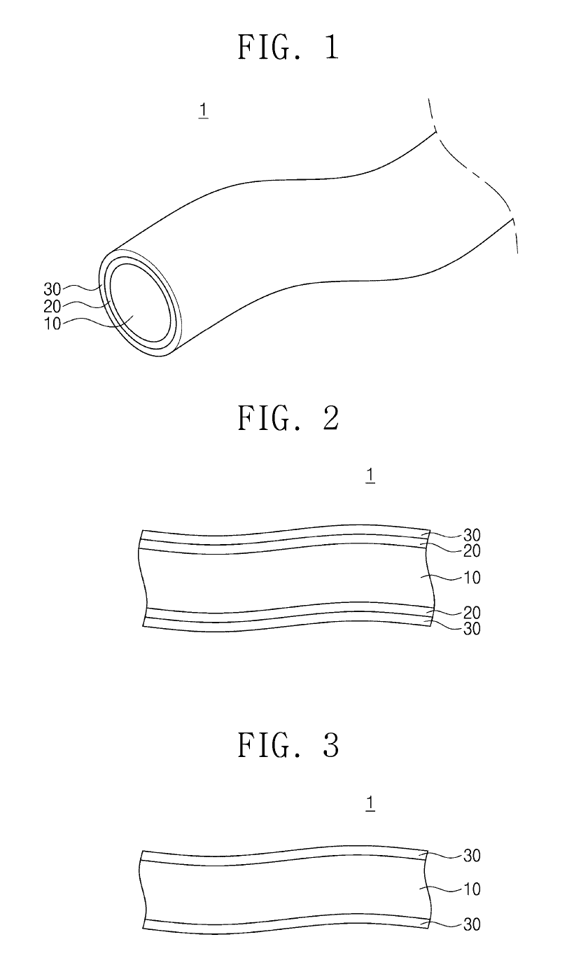

FIG. 1 is a partial perspective view for explaining an example of a sensor fiber according to exemplary embodiments of the present inventive concepts. FIG. 2 is a cross-sectional view for explaining the sensor fiber of FIG. 1. FIG. 3 is a cross-sectional view for explaining other example of a sensor fiber according to exemplary embodiments of the present inventive concepts. In the embodiments of the present inventive concept, a sensor fiber may also be applicable to a chemical sensor. For example, the sensor fiber may be configured to detect or sense gases or liquids.

Referring to FIGS. 1 and 2, a core fiber 10 may be provided. In this description, the term of fiber may mean a thread for making textile products such as a woven fabric, a braided fabric, a lace fabric, a multifilament fabric, and so on. In some embodiments, the core fiber 10 may include at least one of a polymer fiber, a natural fiber, a metal fiber, an inorganic fiber, or a composite fiber thereof. For example, the natural fiber may include a cotton fiber, a hemp fiber, a wool fiber, or a silk fiber. For example, the polymer fiber may include a derivative such as rayon, nylon, cellulose, acryl, polyester, polyethylene, polypropylene, polyethyleneterephthalate, polypropyleneterephthalate, polyacrylonitrile, polyethylenenaphtalate, polyethersulfone, polyetheretherketone, polyphenylenesulfide, polyvinylidenefluoride, or a copolymer thereof. For example, the metal fiber may include an iron alloy, a nickel-chromium alloy, a tungsten alloy, or a copper alloy. For example, the inorganic fiber may include a glass fiber or a rock wool-slag fiber. The aforementioned materials are, however, merely an example, and the present inventive concept is not limited thereto. The core fiber 10 may be properly chosen depending on various detection environments and a kind of target sample intended to detect by the sensor fiber 10, in consideration of characteristics such as strength, elasticity, contractibility, heat resistance, or chemical resistance.

An adhesive member 20 may be adhered to a surface of the core fiber 10. The adhesive member 20 may be coated to surround a circumferential surface of the core fiber 10. The adhesive member 20 may include polydopamine, BSA (Bovine Serum Albumin), beta-amyloid, polylysine, chitosan, or any other polymer adhesives. The adhesive member 20 may be provided to enhance adhesion between the core fiber 10 and a detection member 30 which will be discussed below. As such, the sensor fiber 1 may have enhanced bending-durability and washing-durability. In other embodiments, as shown in FIG. 3, adhesive member 20 may not be provided in the sensor fiber 1. In other words, a later-discussed detection member 30 may be in direct contact with the surface of the core fiber 10.

A detection member 30 may be adhered to a surface of the adhesive member 20. In detail, the detection member 30 may be coated to surround a circumferential surface of the adhesive member 20. The detection member 30 may extend along a longitudinal direction of the core fiber 10. The detection member 30 may include a conductive material, and thereby provide an electrical path between opposite ends of the core fiber 10. The detection member 30 may include a transition metal chalcogenide. For example, the transition metal chalcogenide may include molybdenum sulfide (MoS₂), tungsten sulfide (WS₂), or a two-dimensional transition metal chalcogenide. The transition metal chalcogenide may have an electron density distribution that can be changed when the detection member 30 adsorbs a substance (or the target sample). That is, the transition metal chalcogenide may have an electrical resistance that is changed due to adsorption of the target sample.

The detection member 30 may further comprise a metal nanoparticle, a graphene, a graphene derivative, a metal oxide, or a conductive polymer. To put it another way, the detection member 30 may be a mixture of the transition metal chalcogenide and one of the metal nanoparticle, the graphene, the graphene derivative, the metal oxide, and the conductive polymer. For example, the metal nanoparticle may include tin oxide (SnO₂), zinc oxide (ZnO), or titanium oxide (TiO₂). For example, the graphene derivative may be graphene including a carboxyl group or an amino group. At least one of the metal nanoparticle, the graphene, the graphene derivative, the metal oxide, or the conductive polymer may be provided for easy detection of the target sample, or alternatively may not be provided as needed.

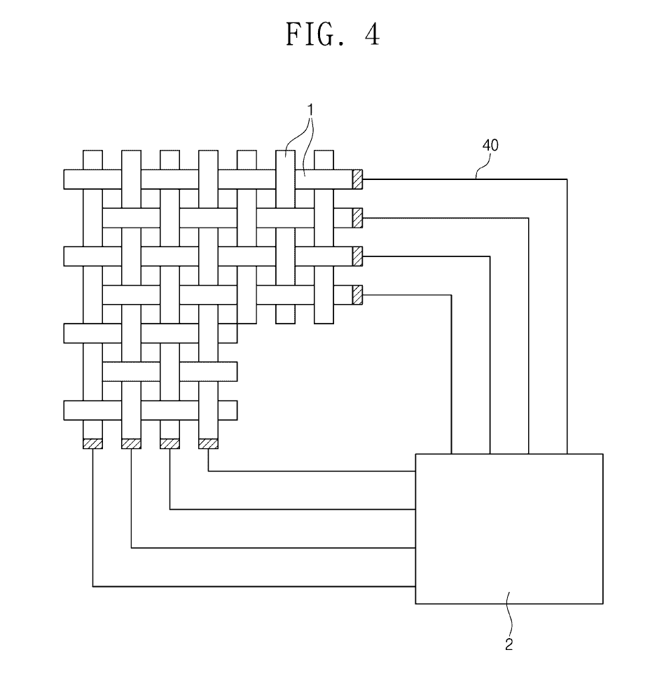

The present inventive concept provides a fabric-based gas sensor system in which the sensor fiber 1 is employed. FIG. 4 is a schematic diagram for explaining a fabric-based gas sensor system using a sensor fiber according to exemplary embodiments of the present inventive concepts. Referring to FIG. 4, a fabric-based gas sensor system may include a controller 2 and the sensor fiber 1 discussed above.

The sensor fiber 1 may be provided. The sensor fiber 1 may be provided in plural, and the plurality of sensor fibers 1 may be woven into a textile structure. For example, the plurality of sensor fibers 1 may be interwoven to have a woven fabric shape. Although FIG. 4 shows that the plurality of sensor fibers 1 are provided in a woven fabric shape, the sensor fibers 1 may be provided in a braided fabric shape or a non-woven fabric shape.

A controller 2 may be connected to opposite distal ends of the sensor fiber 1. For example, the controller 2 may include a measuring unit such as a resistance meter controller capable of measuring variation in electrical signal. An interconnect member 40, such as a lead line or an electrode, may be provided to electrically connect the controller 2 to the detection member 30 of the sensor fiber 1. The controller 2 may measure variation in resistance of the sensor fiber 1. In detail, when a sample intended to detect is absorbed on the detection member 30 of the sensor fiber 1, the detection member 30 may experience variation in its resistance. At this time, based on the variation in resistance on the opposite distal ends of the sensor fiber 1, the controller 2 may ascertain that the adsorbed sample is detected or sensed.

FIG. 4 shows that the plurality of sensor fibers 1 are provided in the woven fabric shape, but the present inventive concepts is not limited thereto. The fabric-based gas sensor system may include a single sensor fiber 1, as needed.

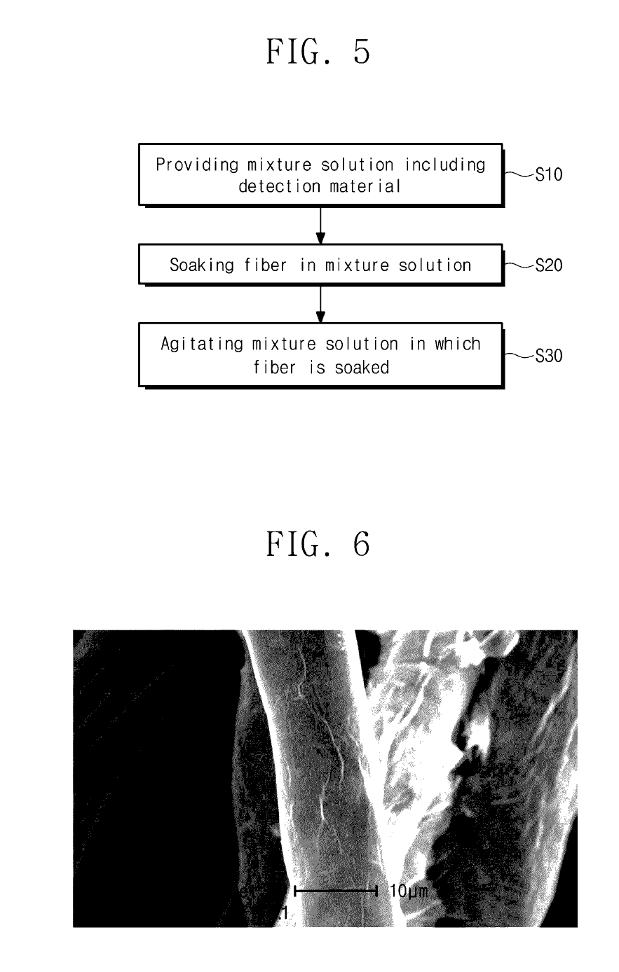

It will be hereinafter described about a method of fabricating a sensor fiber according to exemplary embodiments of the present inventive concept. FIG. 5 is a flow chart for explaining a method of manufacturing a sensor fiber according to exemplary embodiments of the present inventive concepts.

Referring to FIG. 5, a mixture solution may be provided to include a detection member (S10). The mixture solution may be obtained by intermingle the detection member with a first solvent. The first solvent may include water, ethanol, methanol, or isopropyl alcohol. The detection member may be present in concentration of about 0.01 mg/mL to about 10 mg/mL of the mixture solution. The detection member may include a transition metal chalcogenide. For example, the transition metal chalcogenide may include molybdenum sulfide (MoS₂) or tungsten sulfide (WS₂).

An individual additive may be added to the mixture solution. The additive may be one of a metal nanoparticle, a graphene, a graphene derivative, a metal oxide, and a conductive polymer. In this case, the detection member may be formed as a mixture of the transition metal chalcogenide and the additive. The detection member may therefore be a mixture of the transition metal chalcogenide and one of the metal nanoparticle, the graphene, the graphene derivative, the metal oxide, and the conductive polymer. The additive may be present in concentration of about 0.01 mg/mL to about 10 mg/mL of the mixture solution. The additive may be added to easily detect a target gas. In other embodiments, as needed, no additive may be added to the mixture solution.

A core fiber may be submerged in the mixture solution (S20). The core fiber may be freely or fixedly by a tool such as a clamp, disposed in the mixture solution. The core fiber may be provided in plural as needed. For example, a plurality of core fibers may be provided interwoven into a woven fabric shape, or alternatively may be provided as a braided fabric or non-woven fabric shape. The core fiber may include at least one of a polymer fiber, a natural fiber, a metal fiber, an inorganic fiber, or a composite fiber thereof. In other embodiments, the core fiber may be submerged in the first solvent in advance, and then the detection member may be dissolved in the first solvent.

The core fiber may be cleaned before submerged in the first solvent, or may be surface treated to provide a good surface to which the detection member is easily adhered. In some embodiments, an adhesive member may be coated on the surface of the core fiber. For example, the core fiber may be provided thereon with an adhesive solution including the adhesive member. The adhesive solution may be obtained by intermixing the adhesive member with a second solvent. The second solvent may include water or Tris-buffer. In this case, the adhesive member may be present in concentration of about 0.01 mg/mL to about 10 mg/mL of the adhesive solution. The adhesive member may include polydopamine, BSA (Bovine Serum Albumin), beta-amyloid, polylysine, chitosan, or any other polymer adhesives. Thereafter, the core fiber may be submerged in the adhesive solution, and stirring may be performed on the adhesive solution in which the core fiber is submerged. The stirring may force the adhesive solution to enter between the core fibers. The stirring of the adhesive solution may be carried out at about 1 hour to about 24 hours. After that, distilled water or ethanol may be employed to clean the core fiber on which the adhesive member is coated. In other embodiments, no adhesive member may be coated on the surface of the core fiber.

Stirring may be performed on a mixture solution in which the core fiber is submerged (S30). The stirring of the mixture solution may be executed to uniformly coat the detection member on the surface of the core fiber. For example, the stirring may force the mixture solution to enter between the core fibers. The stirring of the mixture solution may be carried out at about 1 hour to about 24 hours. Thereafter, when the detection member is sufficiently coated and fixed onto the core fiber, the core fiber may be withdrawn from the mixture solution. In some embodiments, a cleaning process may be performed on the withdrawn core fiber. The cleaning process may be carried out to remove a non-fixed detection member from the core fiber. For example, the cleaning process may be executed using distilled water or ethanol. Thereafter, a drying process may be performed on the core fiber.

In the embodiments mentioned above, the detection member may be coated on the core fibers that have already been fabricated to have a woven, braided, or non-woven fabric structure; however this process order is merely illustrative and should not be considered a limitation to the present inventive concept. For example, the detection member may be coated on strands of a plurality of core fibers in advance, and then the core fibers may be withdrawn to perform a process of making a woven, braided, or non-woven fabric structure.

It will be hereinafter described about an experimental example of fabricating a sensor fiber.

### Experimental Example

An adhesive member was coated on a cotton fiber, as described below. An adhesive solution was obtained by intermixing polydopamine with Tris-buffer solvent of which pH is about 8.5. The polydopamine was intermixed in concentration of about 2 mg/mL with respect to the Tris-buffer solvent. A cotton fiber was submerged in the adhesive solution, and stirring was performed on the adhesive solution including the cotton fiber submerged therein for about 24 hours at a room temperature (e.g., about 25° C.). Thereafter, the cotton fiber was withdrawn from the adhesive solution and then cleaned with distilled water. The cotton fiber was cleaned again with ethanol.

A detection member was coated on the cotton fiber on which the adhesive member was coated, as discussed below. A mixture solution was obtained by intermixing molybdenum sulfide (MoS₂) and graphene oxide with water solvent. Each of the molybdenum sulfide and graphene oxide was intermixed in concentration of about 1 mg/mL with respect to the water solvent. The cotton fiber was submerged in the mixed solution, and stirring was performed on the mixed solution including the cotton fiber submerged therein for about 24 hours at a room temperature. The cotton fiber, on which the detection member (i.e., molybdenum sulfide and graphene oxide) is coated, may be withdrawn from the mixture solution and then cleaned with distilled water. The cotton fiber may be cleaned again with ethanol, and then dried. After that, a process may be performed to reduce the graphene oxide. The graphene oxide may be reduced by a vapor reaction at a room temperature using HI—AcOH (hydriodic acid with acetic acid (HI—AcOH). In detail, the core fiber on which the detection member is coated was reacted for about 24 hours in a hermetically sealed glass reactor including therein a mixture solution obtained by intermixing hydriodic acid (HI) of about 1 mL with acetic acid (C₂H₄O₂) of about 3 mL. FIG. 6 is a SEM photograph of a sensor fiber manufactured by the experimental example. As seen in FIG. 6, a sensor fiber, whose diameter is about 12 μm, was fabricated to have the detection member coated thereon.

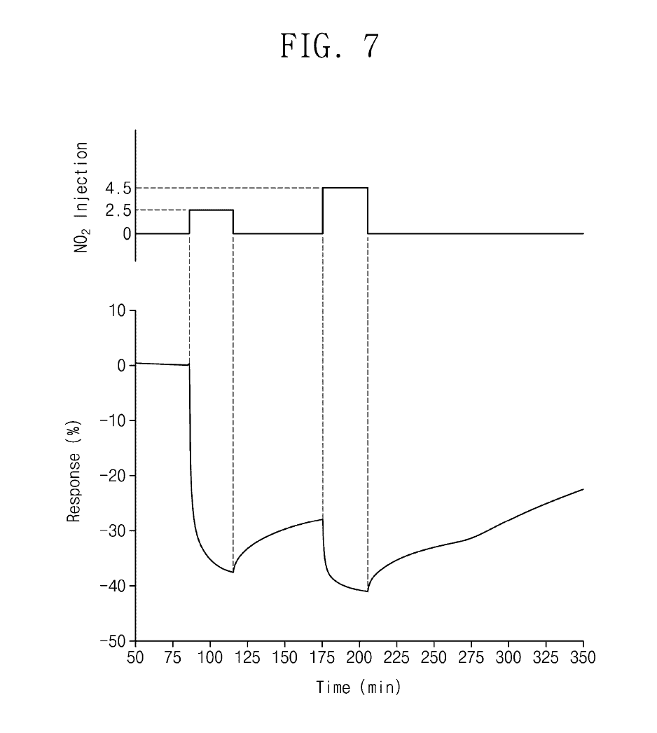

The sensor fiber fabricated by the experimental example was used to measure its resistance change depending on variation in concentration of NO₂ gas. A mass flow controller (MFC) was utilized to measure detection sensibility of the sensor fiber with respect to NO₂ gas, while maintaining a gas flow rate of about 100 sccm under air atmosphere. FIG. 7 is a graph showing NO₂ detection result of a sensor fiber fabricated by an experimental example. Referring to FIG. 7, the graph may show a NO₂ gas detection signal indicating that the sensor fiber exhibits about 37% reduced resistance when NO₂ gas of concentration of 2.5 ppm flows over the sensor fiber in comparison when only air flows over the sensor fiber. The NO₂ gas detection signal may also indicate that, when NO₂ gas of concentration of about 4.5 ppm flows, the sensor fiber exhibits a dramatically reduced resistance shortly after the NO₂ gas injection. The NO₂ gas detection signal may additionally verify that resistance of the sensor fiber recovers to rise toward its initial value at the same time when NO₂ gas injection stops in the two cases.

According to the present inventive concept, the sensor fiber may have a flexible fiber shape that is effectively applicable for wearable devices. The sensor fiber may work at a room temperature, and possibly washed due to its enhanced durability. In addition, the sensor fiber may be fabricated through simplified processes such as submerging and stirring.

Although the present invention has been described in connection with the embodiments of the present inventive concept illustrated in the accompanying drawings, it will be understood by one of ordinary skill in the art that variations in form and detail may be made therein without departing from the spirit and essential feature of the present inventive concept. The above disclosed embodiments should thus be considered illustrative and not restrictive.

What is claimed is:

1. A method of manufacturing a sensor fiber, the method comprising:
providing a mixture solution including a detection member;
submerging a core fiber in the mixture solution; and
coating the detection member on a surface of the core fiber by stirring the mixture solution in which the core fiber is submerged,
wherein the detection member comprises a transition metal chalcogenide.

2. The method of claim 1, wherein the transition metal chalcogenide comprises molybdenum sulfide (MoS₂) or tungsten sulfide (WS₂).

3. The method of claim 1, wherein the core fiber comprises at least one of a polymer fiber, a natural fiber, a metal fiber, an inorganic fiber, or a composite fiber thereof.

4. The method of claim 3, wherein the core fiber is provided in plural, the plurality of core fibers are interwoven into a woven fabric or are braided into a braided fabric.

5. The method of claim 1, wherein the detection member further comprises at least one of a metal nanoparticle, a graphene, a graphene derivate, a metal oxide, or a conductive polymer.

6. The method of claim 1, further comprising, before submerging the core fiber in the mixture solution, coating an adhesive member on the surface of the core fiber,
wherein the detection member is coated on a surface of the adhesive member when the mixture solution is stirred.

7. The method of claim 6, wherein coating the adhesive member on the surface of the core fiber comprises:
providing an adhesive solution that includes the adhesive member;
submerging the core fiber in the adhesive solution; and
coating the adhesive member on the surface of the core fiber by stirring the adhesive solution in which the core fiber is submerged.

8. The method of claim 6, wherein the adhesive member comprises polydopamine, BSA (Bovine Serum Albumin), beta-amyloid, polylysine, chitosan, or any other polymer adhesives.

9. A sensor fiber, comprising:
a core fiber;
an adhesive member surrounding a surface of the core fiber; and
a detection member surrounding a surface of the adhesive member,
wherein the detection member comprises a transition metal chalcogenide.

10. The sensor fiber of claim 9, wherein the transition metal chalcogenide comprises molybdenum sulfide (MoS₂) or tungsten sulfide (WS₂).

11. The sensor fiber of claim 9, wherein the detection member further includes at least one of a metal nanoparticle, a graphene, a graphene derivate, a metal oxide, or a conductive polymer.

12. The sensor fiber of claim 9, wherein the adhesive member comprises polydopamine, BSA (Bovine Serum Albumin), beta-amyloid, polylysine, chitosan, or any other polymer adhesives.

13. The sensor fiber of claim 9, wherein the core fiber comprises at least one of a polymer fiber, a natural fiber, a metal fiber, an inorganic fiber, or a composite fiber thereof.
